(19) United States
(12) Patent Application Publication
(10) Pub. No.: US 2004/0170654 A1
(43) Pub. Date: Sep. 2, 2004

(54) Enhanced parmacokinetic profile of hydrophobic dopamine agonists administered to the dermis

(76) Inventor: Thomas C. Pinkerton, Kalamazoo, MI (US)

Correspondence Address: Harness Dickey & Pierce, Suite 400, 7700 Bonhomme Avenue, St Louis, MO, 63105, US

(21) Appl. No.: 10/480,973

(22) PCT Filed: Jun. 24, 2002
(86) PCT No.: PCT/US02/19918

Related U.S. Application Data
(63) Continuation-in-part of application No. 09/897,801, filed on Jun. 29, 2001.

(51) Int. Cl. A61K 31/542; A61K 31/538; A61K 31/498

(52) U.S. Cl. 424/400; 514/224.5; 514/230.2; 514/250; 514/234.2

(57) ABSTRACT

A method for systemic administration of a hydrophobic dopamine agonist to a mammal is disclosed. The method involves delivering the hydrophobic dopamine agonist to the dermis of the mammal whereby improved systemic absorption is obtained compared to absorption produced upon delivering the substance subcutaneously by bolus administration.

### CROSS REFERENCE TO RELATED APPLICATIONS

This application is a continuation-in-part of U.S. application Ser. No. 09/897,801 filed Jun. 29, 2001.

### BACKGROUND OF THE INVENTION

(1) Field of the Invention

The present invention relates to administration of substances into the dermis and, more particularly, to methods, compositions and devices for administration of hydrophobic substances into the dermis. Such administration results in systemic absorption, which is improved as compared to absorption obtained following subcutaneous administration.

(2) Description of the Related Art

The importance of administering pharmaceutical substances such as diagnostic agents or drugs in a manner that results in good absorption and biologic effectiveness has long been recognized. Such biological effectiveness is dependent upon both systemic presentation of the substance as reflected in pharmacokinetic parameters and drug action as reflected in pharmacodynamic measurements (for review see Cawello et al, J. Clin. Pharmacol. 37:65S-69S, 1997; Wasan et al., Arch. Med. Res. 24:395-401 1993; Ratain, Semin. Oncol. 19:8-13, 1992).

Hydrophobic substances present a particular challenge in achieving desired biological effects due to difficulties in the preparation of delivery formulations coupled with the significant distribution of these substances into adipose tissue and storage in such tissue. (Steiner et al., Drug Metab. Dispos. 19:8-14, 1991; xie et al, Drug Metab. Dispos. 19:15-19, 1991; Hough et al. Life Sci. 58:119-122, 1996). As a result, hydrophobic substances often show no more than limited systemic absorption following most conventional routes of administration. Commonly used routes for systemic administration have involved subcutaneous, intramuscular or intravenous delivery. All of these routes of administration can be considered transdermal administration, i.e. delivery of substances through the skin to a site beneath the skin. Typically, conventional needles have been used for delivering substances transdermally although other approaches have also been used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to at the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical substances. The dermis, however, has rarely been targeted as a site for administration of substances, and this may be due, at least in part, to the difficulty of precise needle placement into the dermis. Furthermore, even though the dermis, in particular, the papillary dermis has been known to have a high degree of vascularity, it has not heretofore been appreciated that one could take advantage of this high degree of vascularity to obtain an improved absorption profile for hydrophobic substances compared to that achieved following subcutaneous administration. This is because small drug molecules are typically rapidly absorbed after administration into the subcutaneous tissue which has been far more easily and predictably targeted than the dermis has been. On the other hand, hydrophobic substances as well as large molecules such as proteins are typically not rapidly or entirely absorbed following subcutaneous administrations. Because hydrophobic substances tend to partition into subcutaneous adipose tissue the absorption of these substances into the vascular system would be expected to be limited following subcutaneous administration (Walder, Immunopharmacol. Immunotoxicol. 13:101-119, 1991). Large proteins would also be expected to be slowly absorbed following subcutaneous administration and bioavailability has often been reported to be highly variable and incomplete (Porter et al., Adv. Drug. Deliv. Rev. 50:157-171, 2001). Furthermore, hydrophobic proteins can show poor absorption following subcutaneous administration measured by standard pharmacokinetic parameters, as compared to values obtained for proteins which are not hydrophobic. (see for example Thomsen et al., Pharmacol. Toxicol. 74:351-358, 1994). In spite of this, hydrophobic substances have not typically been administered into the dermis.

Numerous reports in the literature have reported on what has been described as “intradermal” administration. Such references, however, have often used the term “intradermal” according to its commonly used meaning of “intracutaneous”, i.e. within the substance of the skin. This would including, primarily, subcutaneous tissue. In other references, so called “intradermal” placement of injected substances is intended to achieve no more than local administration of the substance and no attempt is made to achieve systemic bioavailability of the injected substances.

One such approach for achieving local administration of substances has been routinely used in the Mantoux tuberculin test. In this procedure, a purified protein derivative is injected at a shallow angle to the skin surface using a 27 or 30 gauge needle (Flynn et al, Chest 106: 1463-5, 1994). A degree of uncertainty in placement of the injection can, however, result in some false negative test results. Moreover, the test has involved a localized injection to elicit a response at the site of injection and the Mantoux approach has not led to the injection of substances into the dermis for the purpose of achieving systemic delivery of substances.

Similarly, local, “intradermal” injections of anesthetic drugs have been used to diminish the pain associated with i.v. catheter insertion and with suturing of lacerations (see for example Criswell et al., Anaesthesia 46:691-692, 1991; Anderson et al., Ann. Emerg. Med. 19:519-22, 1990). Such usage of local anesthetic drugs is, however, intended to be localized at the site of injection and systemic delivery of the local anesthetic agents is not obtained.

Some groups have reported on systemic administration by what has been characterized as “intradermal” injection. In one such report, a comparison study of subcutaneous and what was described as “intradermal” injection was performed (Autret et al, Therapie 46:5-8, 1991). The pharmaceutical substance tested was calcitonin, a protein of a molecular weight of about 3600, which is highly water soluble in the injectable form, was used. Similarly, Bressolle et al. administered the water soluble substance, sodium ceftazidime in what was characterized as “intradermal” injection (Bressolle et al. J. Pharm. Sci. 82:1175-1178, 1993). Neither of these studies provide any predictive information about absorption of hydrophobic substances following administration to the dermis.

Another group reported on what was described as an “intradermal” drug delivery device (U.S. Pat. No. 5,997,501 to Gross et al.). Injection was indicated to be at a slow rate and the injection site was intended to be in some region below the epidermis, i.e., the interface between the epidermis and the dermis or the interior of the dermis or subcutaneous tissue. This reference taught administration with a particular device by infusion at slow rates which would not be expected show any improved systemic absorption as measured by pharmacokinetic parameters, compared that obtained following subcutaneous administration. This is because at slow infusion rates, the rate of infusion would be the rate limiting determinate of absorption and not tissue absorption barriers. As such, dermal absorption would not be expected to be greater than subcutaneous absorption.

Thus there remains a continuing need for effective methods and devices for administration of hydrophobic substances in a manner that achieves rapid and complete systemic absorption of the compounds.

### SUMMARY OF THE INVENTION

Accordingly, the inventor herein has succeeded in discovering an approach for administration of hydrophobic substances in which improved absorption is achieved compared to that produced upon subcutaneous administration of the substances. The improved absorption is indicated by an improvement in at least one pharmacokinetic or pharmacodynamic parameter. The approach involves selectively delivering the hydrophobic substances into the dermis. Delivery of the hydrophobic substance is to the dermis or into a region in close proximity to the dermis such that absorption takes place predominantly in the dermis. Preferably, the hydrophobic substance is administered by bolus, i.e. within a short period of time of about 10 minutes to about 15 minutes or less and, more preferably within 2 minutes or less. Surprisingly, such delivery to the dermis results in an improvement in pharmacokinetic and/or pharmacodynamic measurements as compared to what is produced upon subcutaneous administration.

Thus, the present invention provides, in one embodiment, a method for systemic administration of a substance to a mammal. Preferably, the mammal is a human although companion animals such as dogs and cats, farm animals such as pigs and cows, exotic animals such as zoo animals and the like are also included within the scope of the present invention.

The term “hydrophobic” as used with respect to a substance administered into the dermis or subcutaneous tissue, is intended to mean that the substance tends to preferentially partition into lipophilic compartments such as can be found in subcutaneous adipose tissues rather than into aqueous extracellular fluids. The hydrophobicity of a substance can be assessed by standard methods such as, for example, by determining the oil-water distribution coefficient, preferably, the n-octanol/water distribution coefficient (see for example, Buchwald, Curr Med Chem 5:353-380, 1998). Values are typically expressed as the hydrophobicity or log value of partition coefficient, logP, under appropriate physiologic conditions. Such conditions will depend upon the conditions of the target region of administration in the mammal including temperature, pH, concentration and the like. Also, log of octanol-water partition coefficients can be estimated by using a variety of calculation programs, such as the one developed by Syracuse Research Corporation (Meylan and Howard, J. Pharm. Sci. 84: 83-92, 1995).

A threshold value of logP_{oct} which correlates with partitioning into adipose tissue is between 1.0 and 2.0 as has been shown by Steiner et al. for barbiturate compounds (Steiner et al., Drug Metabolism and Disposition 19:8-14, 1991; see, in particular, FIG. 4). This has also been shown for a number of basic drugs (Betschart et al, Xenobiotica 18:113-121, 1988; see, in particular, FIG. 2). Thus the hydrophobic substances of the present invention have a threshold logP value greater than 1.00 preferably, at least about 1.5 or greater. A linear relationship has been shown between adipose tissue uptake and logP_{oct} values up to 5 and greater for some compounds (Betschart et al., supra). Thus, in certain embodiments, it is desirable that the hydrophobic compound have a logP_{oct} of greater than 1.5, i.e., at least about 2.0 or greater, at least about 2.5 or greater, at least about 3.0 or greater, at least about 3.5 or greater, at least about 4.0 or greater, or at least about 5.0 or greater.

The hydrophobic substances of the present invention can be small molecular drugs or diagnostic agents or large molecules such as proteins, polysaccharides or other polymeric compounds. The hydrophobic substances of the present invention include the non-limiting examples, anticonvulsant hydantoins, barbituric acids, HV protease inhibitors, antiviral nucleosides, tricyclic nitrogen-containing compounds for central nervous system and sexual dysfunction conditions as well as numerous other hydrophobic substances. The invention is particularly applicable to tricyclic nitrogen-containing compounds useful in treating sexual dysfunction in men and women as disclosed in International Patent Publication No. WO 00/40226. Compounds of this class were earlier disclosed in U.S. Pat. No. 5,273,975 (both WO 00/40226 and U.S. Pat. No. 5,273,975 are incorporated in their entireties, by reference).

Such compounds are of the formula (I)

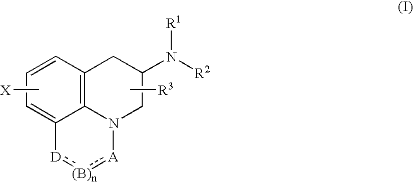

or pharmaceutically acceptable salts thereof, wherein

R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;

X is H, F, Cl, Br, I, OH, C₁₋₆ alkyl or alkoxy, CN, carboxamide, carboxyl or (C₁₋₆ alkyl)carbonyl;

A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;

B is CH, CH₂, CHF, CHCl, CHBr, CHI, C═O, N, NH or NCH₃, and n is 0 or 1; and

D is CH, CH₂, CHF, CHCl, CHBr, CHI, C═O, O, N, NH or NCH₃;

with various provisos as indicated in WO 00/40226.

Preferred compounds useful in methods of the present invention are those disclosed generically or specifically in above-cited U.S. Pat. No. 5,273,975. Especially preferred compounds are those of formula (II)

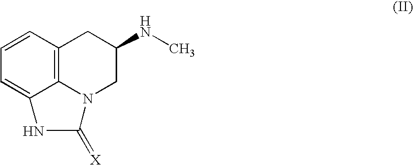

wherein X is O or S.

The improved systemic absorption produced by delivery to the dermis can be measured by any one of a number of standard pharmacokinetic and/or pharacodynamic parameters such as, for example, increase in bioavailability, decrease in Tₘₐₓ increase in Cₘₐₓ, decrease in T_{lag}, or the like. By bioavailability is meant the total amount of a given dosage that reached the blood compartment. This is generally measured as the area under the curve in a plot of concentration vs. time, i.e. AUC. Although, bioavailability theoretically includes the total amount reaching the blood compartment over an infinite time period after dosage, as a practical matter, bioavailability is measured over a finite time interval of several hours after dosing, such as for example, about 2 hours, about 4, hours, about 6 hours, about 8, about 12 hours, about 14 hours or about 24 hours after dosing or longer.

By “lag time” or T_{lag} is meant the delay between the administration of a compound and time to measurable or detectable blood or plasma levels. Although T_{lag} will be dependent upon the sensitivity of the assay method measuring or detecting the blood or plasma levels of a substance, the decrease in T_{lag} is independent of assay method because the same assay method is used to measure blood or plasma levels under the comparator conditions to show the decrease in T_{lag}. For example, the same assay system is used to compare plasma levels after subcutaneous administration and after administration of a substance to the dermis. A shorter time for achieving detectable levels of the substance, following administering into the dermis compared to subcutaneous administration, indicates improved absorption.

Tₘₐₓ is a value representing the time to achieve maximal blood concentration of the compound, and Cₘₐₓ is the maximum blood concentration reached with a given dose and administration method. The time for onset of action is related to T_{lag}, Tₘₐₓ and Cₘₐₓ, inasmuch as all of these parameters influence the time necessary to achieve a blood (or target tissue) concentration necessary to realize a biological effect. Tₘₐₓ and Cₘₐₓ can be determined by visual inspection of graphical results and can often provide sufficient information to compare two methods of administration of a compound. However, numerical values can be determined more precisely by analysis using kinetic models (as described below) and/or other means known to those of skill in the art.

Delivery into the dermis can be with any of a wide variety of devices which produce a cutaneous micropore such as is produced by any solid projection, electromotive force, thermal energy or gas ballistics. Such devices are referenced herein as poration devices, in particular microporation devices, or dermal-access devices. Preferably delivery is through one or more hollow needles although needleless or needle-free ballistic injection of fluids or powders into the ID space, iontophoresis, electroporation, or direct deposition of fluid, solids, or other dosing forms into the skin and the like are also within the scope of the present invention so long at least one cutaneous micropore is established by the delivery device.

The methods of the present invention can involve, in one embodiment, a selective delivery of the hydrophobic substance to the dermis. Such selective delivery involves intentional placement of the substance in the dermis or in the region of the dermis which will or does result in access to and unimpeded absorption of the substance in the dermis as compared to placement in any other region of the skin. The selective delivery can comprise, in whole or in part, a recognition that delivery of the substance is to the dermis. In one aspect of this embodiment, the delivery of the substance to the dermis results in systemic absorption and, preferably, improved systemic absorption is obtained. Such improved systemic absorption can comprise a substantially higher bioavailability and/or a substantially higher Cₘₐₓ, and/or a substantially shorter Tₘₐₓ and/or a substantially shorter T_{lag}. In a variation of the embodiment, the delivery is intended to achieve systemic absorption, and preferably, improved systemic absorption. Such selective delivery to obtain improved systemic absorption can comprise, in whole or in part, measurement of one or more pharmacokinetic parameters showing such improvement.

Among the several advantages achieved by the present invention, therefore, may be noted the provision of a new parenteral route of administration of hydrophobic substances which can achieve systemic delivery of the substances; the provision of a method of administration suitable for obtaining rapid onset of action of hydrophobic substances; the provision of methods suitable for obtaining repeated bolus administrations which mimic the pulsatile, rapid release of natural hormones; the provision of methods which allow intermittent and/or pulsatile administration of hydrophobic hormones or mimetics which avoids any receptor down regulation elicited by continuous blood levels of the hormones; the provision of a mode of administration which achieves a high absorption and bioavailability to allow less active drug to be used at a reduction in cost and a diminished likelihood of side effects; the provision of method of administration of a hydrophobic substance which can achieve higher blood levels than that achieved with subcutaneous administration of the same dose level; the provision of a method of administration which produces increases efficacy of the substance without altering systemic elimination rates and without altering pharmacodynamic effects; the provision of a method which avoids the substance being trapped in subcutaneous tissue lipophilic compartments to produce a depot effect; the provision of a method which is amenable to a regulated dose regimen as a result of rapid absorption of administered substance; and the provision of a method of cutaneous administration which when implemented with a hollow needle device, places the substance directly in the dermis to avoid metabolic degredation and/or immunologic activity which can occur in the epidermis.

### DETAILED DESCRIPTION OF THE INVENTION

In accordance with the present invention, it has been discovered that administration of a hydrophobic substance to the dermis results in improved systemic absorption of the substance.

The hydrophobic substances of the present invention tend to be of low water solubility or to be water insoluble, but soluble in non-polar solvents. Hydrophobicity of a substance can be assessed by standard methods such as, for example, by determining the oil-water distribution coefficient, preferably, the n-octanol/water distribution coefficient (see for example, Buchwald, Curr Med Chem 5:353-380, 1998). The oil-water distribution is the ratio of concentration of a compound in a water-immiscible non-polar solvent phase, such as n-octanol to the concentration in a water phase in contact with the solvent phase. Values are typically expressed as the log value of partition coefficient, logP, under appropriate physiologic conditions. Such conditions will depend upon the conditions of the target region of administration in the mammal including temperature, pH, concentration and the like.

For substances which are ionizable, the pK values or the negative log of the dissociation constant of such substances are a consideration and these values are sometimes determined at the same time that hydrophobicity is determined. This is because the partition coefficient is the ratio of concentration of a substance as the neutral molecule in a water-immiscible solvent to its concentration in an aqueous phase. Therefore, it is important to know the amount of neutral species present and this can be determined the pK of the substance and the pH of the aqueous solution. The pKa is the negative log of the equilibrium constant of an acid and the pKb is the negative log of the equilibrium constant of a base. As a practical matter, an acid having a pKa of one or more units greater than 7.4, i.e. 8.4 or greater, or a base having a pKb of one or more units less than 7.4, i.e. 6.4 or less are each predominantly in the form of the neutral molecule at a physiologic pH of 7.4 and this neutral form will partition substantially into the oil phase in an oil-water distribution test.

Another factor which will influence the measured value of logP, in addition to pH, is the particular non-polar solvent used in the oil phase. Typically, n-octanol is the non-polar solvent because this substance has a carbon to oxygen ratio, which is similar to that of lipid material in animal fats. Thus, the n-octanol partition coefficient is believed to reflect the distribution of a substance administered to a subject into regions of the body containing a significant amount of adipose tissue.

The partition coefficient of a substance can be measured by any of a number of methods known in the art. These include, for illustrative purposes only, potentiometric methods such as with PCA101 of GlpKa(™) devices (Sirius Analytical Instruments, Ltd, East Sussex, UK) which measure both pKa and partition coefficient, filter probe methods (Tomilinson, J. Pharm. Sci 71:602-604, 1982); reverse phase HPLC methods (see for example, Valko et al., Curr. Med. Chem. 8:1137-1146, 2001), flask shaking methods, predictive methods (see for example Buchwald et al., Curr. Med. Chem. 5:353-380, 1998) and the like.

The LogP has been shown to be related to aqueous solubility according to the following equation (Hansch et al., J. Org. Chem. 33:347-350, 1968):

logS_{w}=−1.34logP_{oct}+0.99

where logS_{w} is the molar solubility and logP_{oct} is the water-oil partition coefficient. Using this equation, values for logP_{oct} can be calculated from solubility data.

The hydrophobic substances of the present invention preferably show an n-octanol-water partition coefficient of at least about 1.5 or greater, more preferably at least about 2.0 or greater, and in certain embodiments, preferably at least about 2.5 or greater, at least about 3.0 or greater, at least about 3.5 or greater or at least about 4.0 or greater.

Hydrophobic substances that can be delivered into the dermis in accordance with the present invention are intended to include pharmaceutically or biologically active substances including diagnostic agents, drugs, and other substances which provide therapeutic or health benefits such as for example nutraceuticals.

The hydrophobic substances of the present invention can be small molecular drugs or diagnostic agents or large molecules such as proteins, polysaccharides or other polymeric compounds. The hydrophobic substances of the present invention include the non-limiting examples, anticonvulsant hydantoins, barbituric acids, HIV protease inhibitors, antiviral nucleosides, cyclooxygenase inhibitors, tricyclic nitrogen-containing compounds for central nervous system and sexual dysfunction conditions as well as numerous other hydrophobic substances.

The invention is particularly applicable to tricyclic nitrogen-containing compounds of the formula (I)

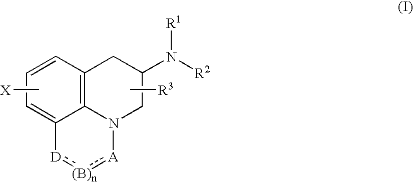

or pharmaceutically acceptable salts thereof, wherein

R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;

X is H, F, Cl, Br, I, OH, C₁₋₆ alkyl or alkoxy, CN, carboxamide, carboxyl or (C₁₋₆ alkyl)carbonyl;

A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;

B is CH, CH₂, CHF, CHCl, CHBr, CHI, C═O, N, NH or NCH₃, and n is 0 or 1; and

D is CH, CH₂, CHF, CHCl, CHBr, CHI, C═O, O, N, NH or NCH₃;

with various provisos as indicated in WO 00/40226.

Especially preferred compounds are those of formula (II)

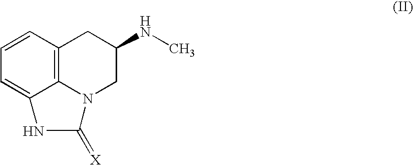

wherein X is 0 (sumanirole) or S (compound II) (see WO 00/40226 and U.S. Pat. No. 5,273,975 which are incorporated in their entireties, by reference). Particularly preferred are compounds in the series useful for treatment of sexual dysfunction, especially, (R)-5,6-dihydro-5-(methylamino)-4H-imidazo[4,5-ij]-quinoline-2(1H)-thione and pharmaceutically acceptable salts as well as sumanirole which is (R)-5,6-dihydro-5-(methylamino)-4H-imidazo[4,5-ij]-quinolin-2(1H)-one and pharmaceutically acceptable salts.

Pharmaceutical acceptability refers to those properties which provide for suitability for administration to a subject including requirements of govermental agencies, patient acceptance and chemical and physical requirements which allow for manufacture, stability, bioavailability in a subject and the like. Pharmaceutically acceptable salts include salts of the following acids: maleic, methansulfonic, hydrochloric hydrobromic, sulfuric, phosphoric, nitric, benzoic, citric, tartaric, fumaric, and the like.

The LogP of (R)-5,6-dihydro-5-(methylamino)-4H-imidazo[4,5-ij]-quinoline-2(1H)-thione was estinated to be 1.62 using logKow software (Syracuse Research Corporation, North Syracuse, N.Y. 13212; see also Meylan and Howard, supra). In accordance with the present invention, this compound would be expected to produce higher Cₘₐₓ values and shorter Tₘₐₓ values upon administration to the dermis than that obtained following subcutaneous administration.

Additional hydrophobic substances within the scope of the present invention include the non-limiting examples of anticonvulsant hydantoins, barbituric acids, HIV protease inhibitors, cyclooxygenase inhibitors, antiviral nucleosides and pinene and its derivatives as shown in Table 1 below.

TABLE 1
LogP Values Of Hydrophobic Substances.

| | LogS_{w} | | |
|---|---|---|---|
| Compound | (moles/L) | LogP_{oct}* | Reference |
| ANTICONVULSANT HYDANTOINS | | | |
| 5,5-diphenyhlhydantoin | −4.10 | 3.80 | Stella et al, J. |
| 3-pentanoyloxymethyl-5,5- | −4.68 | 4.23 | Pharm. Sci, |
| diphenylhydantoin | | | 88: 775-79, 1999 |
| 3-octanoloxymethyl-5,5- | −6.52 | 5.60 | |
| diphenylhydantoin | | | |
| Anethole trithione | −5.80 | 5.07 | |
| dithiolethione | −2.48 | 2.59 | |
| 5-phenyldithiolethione | −5.64 | 4.95 | |
| dimethylthiolethione | −3.42 | 3.29 | |
| BARBITURIC ACIDS | | | |
| 5,5-dimethylbarbituric acid | −1.74 | 2.04 | Prankerd et al Int. |
| 5-Me-5-ethylbarbituric acid | −1.23 | 1.66 | J. Pharm. 112: 1-15, |
| 5-Me-5-allylbartituric acid | −1.16 | 1.60 | 1994 |
| 5-Me-5-phenylbarbituric acid | −2.38 | 2.51 | |
| 5-Me-5-(3-methylbut-2-enyl)barbituric | −2.60 | 2.68 | |
| acid | | | |
| 5,5-diethylbarbituric acid | −1.40 | 1.78 | |
| 5-Et-5-iPr-barbituric acid | −2.15 | 2.34 | |
| 5-Et-5-allyl-barbituric acid | −1.61 | 1.64 | |
| 5-Et-5-phenyl-barbituric acid | −2.32 | 2.47 | |
| 5-Et-5-(3-methylbut-2-enyl)-barbituric | −2.25 | 2.42 | |
| acid | | | |
| 5,5-diphenylbarbituric acid | −4.20 | 3.87 | |
| 5,5-di-iPr-barbituric acid | −2.77 | 2.81 | |
| 5-iPr-5-allyl-barbituric acid | −1.71 | 2.01 | |
| 5-iPr-5-(3-methylbut-2-enyl)-barbituric | −2.59 | 2.67 | |
| acid | | | |
| 5-tBu-5-(3-methylbut-2-enyl)-barbituric | −3.55 | 3.39 | |
| acid | | | |
| 5-diallyl-barbituric acid | −2.08 | 2.29 | |
| 5-allyl-5-phenylbarbituric acid | −2.37 | 2.51 | |
| 5-diethyl-2-thiobarbituric acid | −2.17 | 2.36 | |
| 5-Et-5-(1-methylbutyl)-2-thiobarbituric | −3.68 | 3.49 | |
| acid | | | |
| 5,5-(CH2)2-barbituric acid | −1.89 | 2.15 | |
| 5,5-(CH2)3-barbituricacid | −1.66 | 1.98 | |
| 5,5-(CH2)4-barbituric acid | −2.35 | 3.97 | |
| 5,5-(CH2)5-barbituric acid | −3.06 | 3.02 | |
| 5,5-CH2)6-barbituric acid | −3.17 | 3.10 | |
| 5,5-(CH2)7-barbituric acid | −2.98 | 2.96 | |
| 5,5-(CH2)10-barbituric acid | −4.59 | 4.16 | |
| 5,5-(CH2)5-2-thiobarbituric acid | −3.46 | 3.32 | |
| 5,5-(CH2)11-barbituric acid | −5.80 | 5.07 | |
| HIV PROTEASE INHIBITORS | | | |
| Didanosine | −0.90 | 1.48 | Williams et al., |
| delavirdine | −4.76 | 4.29 | Adv. Drug Del. |
| Efavirenz | −4.57 | 4.15 | Rev. 39: 211-238, |
| Indinavir | −3.94 | 3.68 | 1999 |
| Ritonavir | −5.16 | 4.52 | |
| Amprenavir | −4.00 | 3.72 | |
| Saquinavir | −4.33 | 3.97 | |
| N-(3{(1R)-1-[(6R)-4-hydroxy-2-oxo-6- | −8.00* | 6.71 | log P estimated |
| phenethyl-6-propyl-5,6-dihydro-2H-pyran- | | | using SRC |
| 3-yl]propyl}phenyl)-5-(trifluoromethyl)-2- | | | program |
| pyridinesulfonamide | | | (Meylan, et al. |
| | | | J. Pharm. Sci. 84: |
| | | | 83-92, 1995.) |
| ANTIVIRAL NUCLEOSIDES | | | |
| N2-acetylacyclovir | −1.92 | 2.17 | Kristl, Med.Res. |
| O-acetylacyclovir | −0.86 | 1.38 | Rev. 7: 417-440, |
| N2,O-diacetylacyclovir | −2.70 | 2.75 | 1999. |
| PINENE AND DERIVATIVES | | | |
| a-pinene | −3.66 | 3.47 | Fichan et al., J. |
| B-pinene | −3.91 | 3.66 | Chem. Eng. Data |
| limonene | −3.41 | 3.28 | 44: 773-777, |
| Myrcene | −3.58 | 3.41 | 1999 |
| Borneol | −2.62 | 2.69 | |
| fenchyl_alcohol | −2.27 | 2.43 | |
| pinene_oxide | −2.59 | 2.67 | |
| a-ionone | −3.06 | 3.02 | |
| carveol | −1.88 | 2.14 | |
| linalool | −2.00 | 2.23 | |
| a-terpineol | −1.91 | 2.16 | |
| Carvone | −1.67 | 1.99 | |
| CYCLOOXYGENASE INHIBITORS | | | |
| Celecoxib | −3.66* | 3.47 | log P estimated |
| Valdecoxib | −2.59* | 2.67 | using SRC |
| Parecoxib | −3.16* | 3.10 | program |
| | | | (Meylan, et al. |
| | | | J. Pharm. Sci. 84: |
| | | | 83-92, 1995.) |

*Log P_{oct} was calculated as logP_{oct} = (0.99 - logS_{w})/1.34 except where otherwise indicated.

The pharmacokinetic profile for individual compounds will vary according to the chemical properties of the compounds. For example, compounds which are hydrophobic small molecules having a molecular weight of no more than 1000 Daltons are expected to show significant changes compared to traditional parenteral methods of administration, such as intramuscular, subcutaneous or subdermal injection. Furthermore, compounds which are hydrophobic and relatively large, having a molecular weight of at least 1000 Daltons as well as larger compounds of at least 2000 Daltons, at least 4000 Daltons, at least 10,000 Daltons and larger are expected to show the most significant changes compared to traditional parenteral methods of administration, such as intramuscular, subcutaneous or subdermal injection.

The enhanced absorption profile is believed to be particularly evident for substances which are not well absorbed when injected subcutaneously such as, for example, hydrophobic substances and, in particular, hydrophobic macromolecules. Macromolecules and, in particular, hydrophobic macromolecules are, in general, not well absorbed subcutaneously and this may be due, not only to their size relative to the capillary pore size, it may also be due to their slow diffusion through the interstitium because of their size and hydrophobicity. It is to be understood that hydrophobic macromolecules can possess discrete hydrophobic domains. In contrast, small molecules which are hydrophilic are generally well absorbed when administered subcutaneously and it is possible that no enhanced absorption profile would be seen upon injection into the dermis compared to absorption following subcutaneous administration.

The hydrophobic substances within the scope of the present invention can include conjugates which are covalently linked. Such conjugates can include the non-limiting examples of high or low molecular weight molecules conjugated with polyethylene glycol (PEG) and other polymers (for review, see Veronese, Biomaterials 22:405-417, 2001). Covalent attachment of PEG to a protein can greatly increase the protein half-life in blood. Protein-protein conjugates, i.e. fusion proteins, are also included within the scope of the present invention such as, for example, single-chain Fv (sFv) conjugates with effector proteins (for review, see Huston et al, Int. Rev. Immunol 10:195-217, 1993). Single-chain Fv antibodies can also be conjugated with small molecules such as imaging tags (see for example, Begen et al, Nat. Med. 2:979-984, 1996) and such conjugates are also within the scope of the present invention.

By “improved pharmacokinetics” it is meant that an enhancement of pharmacolinetic profile is achieved as measured, for example, by standard pharmacokinetic parameters such as time to maximal plasma concentration (Tₘₐₓ), the magnitude of maximal plasma concentration (Cₘₐₓ) or the time to elicit a minimally detectable blood or plasma concentration (T_{lag}. By enhanced absorption profile, it is meant that absorption is improved or greater as measured by such pharmacokinetic parameters. The measurement of pharmacokinetic parameters and determination of minimally effective concentrations are routinely performed in the art. Values obtained are deemed to be enhanced by comparison with a standard route of administration such as, for example, subcutaneous administration or intramuscular administration. In such comparisons, it is preferable, although not necessarily essential, that administration into the dermis and administration into the reference site such as subcutaneous tissue, involve the same dose levels, i.e. the same amount and concentration of drug as well as the same carrier vehicle. Administration to the reference site can be at a bolus rate of administration method and/or administration can be at the at the same rate as administration to the dermis whether at a bolus rate of administration or at a slower, infusion rate of administration. Administration to the reference site at a bolus rate of administration is preferred for achieving a comparative enhancement of systemic absorption inasmuch as such bolus administration of hydrophobic substances to the subcutaneous tissue shows a diminished rate of systemic absorption compared to absorption of substances which are hydrophilic or substances which are less hydrophobic than the test substance (see for example, Fuji et al, Exp. Anim. 48:241-246, 1999). Thus, the improvement in systemic absorption as reflected in pharmacokinetic parameters is more pronounced following administration to the dermis as compared to values measured following bolus subcutaneous administration.

Comparison can also be at the same rate of administration in terms of amount and volume per unit time. Thus, for example, administration of a given pharmaceutical substance into the dermis at a concentration such as 100 μg/ml and rate of 100 μL per minute over a period of 5 minutes would, preferably, be compared to administration of the same pharmaceutical substance into the subcutaneous space at the same concentration of 100 μg/ml and rate of 100 μL per minute over a period of 5 minutes.

Administration of a hydrophobic substance to the dermis is intended to mean that the substances is placed in such a manner that the substance readily reaches the richly vascularized papillary dermis and is rapidly absorbed into the blood capillaries and/or lymphatic vessels to become systemically bioavailable. Such can result from placement of the substance in the upper region of the dermis, i.e. the papillary dermis or in the upper portion of the relatively less vascular reticular dermis such that the substance readily diffuses into the papillary dermis.

Mammalian skin contains two layers, as discussed above, specifically, the epidermis and dermis. The epidermis is made up of five layers, the stratum corneum, the stratum lucidum, the stratum granulosum, the stratum spinosum and the stratum germinativum and the dermis is made up of two layers, the upper papillary dermis and the deeper reticular dermis. The thickness of the dermis and epidermis in humans varies from individual to individual, and within an individual, at different locations on the body. For example, it has been reported that the epidermis varies in thickness from about 40 to about 90 μm and the dermis varies in thickness ranging from just below the epidermis to a depth of from less than 1 mm in some regions of the body to just under 2 to about 4 mm in other regions of the body depending upon the particular study report (Hwang et al., Ann Plastic Surg 46:327-331, 2001; Southwood, Plast. Reconstr. Surg 15:423-429, 1955; Rushmer et al., Science 154:343-348, 1966). The invention herein with respect to administration to humans, encompasses delivery of substances to the dermis at any desired location on the body. Thus the depth of placement of the substance will depend upon the depth of the dermis at the desired location. Such placement may be, for example, from up to about 1 mm in certain instances for abdominal skin (Hwang et al., supra) or up to about 4 mm in certain instances for skin of the back (Rushmer et al., supra).

For most areas of human skin, it is preferred that a substance be placed predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of macromolecular and/or hydrophobic substances. Placement of the substance predominately at greater depths and/or into the lower portion of the reticular dermis is believed to result in the substance being slowly absorbed in the less vascular reticular dermis or in the subcutaneous region either of which would result in reduced absorption of macromolecular and/or hydrophobic substances. The controlled delivery of a substance to the dermis below the papillary dermis in the reticular dermis, but sufficiently above the interface between the dermis and the subcutaneous tissue, should enable an efficient (outward) migration of the substance to the (undisturbed) vascular and lymphatic microcapillary bed (in the papillary dermis), where it can be absorbed into systemic circulation via these microcapillaries without being sequestered in transit by any other cutaneous tissue compartment.

The present invention provides a method for therapeutic treatment by delivery of a hydrophobic drug or other substance to a human or non-human animal subject by directly targeting the dermis, wherein the drug or substance is administered through any one of a variety of dermal-access devices. Substances administered according to the methods of the invention have been found to exhibit improved pharmacokinetic parameters, and more clinically desirable than that observed for the same substance administered by subcutaneous injection, i.e. by bolus subcutaneous administration.

The microporation device or dermal-access device used for administration to the dermis according to the invention is not critical as long as it penetrates the skin of a subject to the desired targeted depth to the dermis without passing through the dermis to the subcutaneous tissue. In most cases, the device will penetrate the skin and to a depth of about 0.5-2 mm. The dermal-access means may comprise conventional injection needles, catheters or microneedles of all known types, employed singularly or in multiple needle arrays. The dermal-access means may comprise needleless devices including ballistic injection devices. The terms “needle” and “needles” as used herein are intended to encompass all such needle-like structures The term microneedles as used herein are intended to encompass structures smaller than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes.

Microporation or dermal-access devices also include ballistic fluid injection devices, powder-jet delivery devices, piezoelectric, electromotive, electromagnetic assisted delivery devices, gas-assisted delivery devices, of which directly penetrate the skin to provide access for delivery or directly deliver substances to the targeted location within the dermal space. The targeted depth of delivery of substances by the dermal-access means may be controlled manually by the practitioner, or with or without the assistance of indicator means to indicate when the desired depth is reached. Preferably however, the device has structural means for controlling skin penetration to the desired depth within the dermis. This is most typically accomplished by means of a widened area or hub associated with the shaft of the dermal-access means that may take the form of a backing structure or platform to which the needles are attached. The length of microneedles as dermal-access means are, preferably, less than 2 mm length. They may be used in the invention as individual single microneedles or in an assembly of multiple microneedles in linear or two-dimensional arrays so as to increase the rate of delivery or the amount of substance delivered in a given period of time. Microneedles may be incorporated into a variety of devices such as holders and housings that may also serve to limit the depth of penetration. The dermal-access devices of the invention may also incorporate reservoirs to contain the substance prior to delivery or pumps or other means for delivering the drug or other substance under pressure. Alternatively, the device housing the dermal-access devices may be linked externally to such additional components.

Microneedles suitable for administration to the dermis may, for example, have the dimensions of about 250 micron outer diameter, and less than 2 mm exposed length. The microneedles can be constructed of steel, other metals such as copper, nickel, titanium or mixtures thereof, silicon, ceramic, plastic, or any suitable material or combinations thereof.

The intravenous-like pharmacokinetics are achieved by administering a substance to the dermis in intimate contact with the capillary microvasculature and lymphatic microvasculature of the papillary dermis. In should be understood that the terms microcapillaries or capillary beds of the dermis are intended to refer to either vascular or lymphatic drainage pathways within the dermis.

While not intending to be bound by any theoretical mechanism of action, it is believed that the rapid absorption observed upon administration into the dermis is achieved as a result of the rich plexuses of blood and lymphatic vessels in the dermis. However, the presence of blood and lymphatic plexuses in the dermis would not by itself be expected to produce an enhanced absorption of hydrophobic substances because the substances tend to partition into lipophilic compartments in a depot fashion to thereby diminish their availability for absorption. The enhanced absorption observed upon administration of a hydrophobic substance to the dermis may, however, result from the lack of fat cells and, hence, the substantial absence of lipophilic compartments in the dermis. Another possible contribution to the unexpected enhanced absorption achieved upon delivery of hydrophobic substances to the dermis may result from an increase in blood flow and capillary permeability caused by injection into the dermis. For example, it is known that a pinprick insertion to a depth of 3 mm produces an increase in blood flow and this has been postulated to be independent of pain stimulus and due to tissue release of histamine (Arildsson et al., Microvascular Res. 59:122-130, 2000). This is also consistent with the observation that an acute inflammatory response elicited in response to skin injury produces a transient increase in blood flow and capillary permeability (see Physiology, Biochemistry, and Molecular Biology of the Skin, Second Edition, L. A. Goldsmith, Ed., Oxford Univ. Press, New York, 1991, p. 1060; Wilhem, Rev. Can. Biol. 30:153-172, 1971). At the same time, the injection into the dermis would be expected to increase interstitial pressure. It is known that increasing interstitial pressure from values from a normal value of about −7 mmHg to about +2 mmHg distends lymphatic vessels and increases lymph flow (Skobe et al., J. Investig. Dernatol. Symp. Proc. 5:14-19, 2000). Thus, the increased interstitial pressure elicited by injection into the dermis is believed to elicit increased lymph flow and increased absorption of substances injected into the dermis.

The administration methods useful for carrying out the invention include both bolus and infusion delivery of drugs and other substances to humans or animals subjects. A bolus dose is a single dose delivered in a single volume unit over a relatively brief period of time, typically about 10 minutes or less, more preferably about 2 minutes or less. Administration by bolus administration can be by a device suitable for accessing the dermis which also contains a mechanism for propelling the substance into the dermis such as, for example, a needle or microneedle coupled to a syringe driven by a pump. Alternatively, the syringe and needle delivery can be used manually by push while monitoring the injection time, typically about 2 minutes or less, with the second hand of a clock or watch.

Infusion administration comprises administering a fluid at a selected rate that may be constant or variable, over a relatively more extended time period, typically greater than about 10 minutes. To deliver a substance the dermal-access device is placed adjacent to the skin of a subject providing directly targeted access within the dermis and the substance or substances are delivered or administered into the dermis where they can act locally or be absorbed into the bloodstream and be distributed systematically. The dermal-access device may be connected to a reservoir containing the substance or substances to be delivered. Delivery from the reservoir into the dermis may occur either passively, without application of the external pressure or other driving means to the substance or substances to be delivered, and/or actively, with the application of pressure or other driving method. Examples of preferred pressure generating devices include pumps, syringes, elastomer membranes, gas pressure, piezoelectric, electromotive, elecrtomagnetic pumping, or Belleville springs or washers or combinations thereof. If desired, the rate of delivery of the substance may be variably controlled by the pressure-generating means. As a result, the substance enters the dermis and is absorbed in an amount and at a rate sufficient to produce a clinically efficacious result. Clinically efficacious results, as referenced herein, are intended to include both diagnostically and therapeutically useful responses, resulting from administration of a substance or substances.

The hydrophobic substances of the present invention are in a formulation suitable for administration to the dermis. The hydrophobic substance can be in the form of a solution in a non-aqueous vehicle or a vehicle which is a mixture of water and a co-solvent. Non-aqueous vehicles and/or cosolvents include sugars and high molecular weight hydrophylic polymers (see for example, Yalkowsky, Solubility and Solubilization in Aqueous Media, Oxford University Press, New York, 1999). Non-limiting examples of such cosolvents include ethanol, propylene glycol, glycerin, sorbitol, polyethylene glycol 400, polyethylene glycol 4000, poloxamer 188, propylene carbonate, polyvinylpyrrolidone, dimethylisosorbide, N-methylpyrrolidone and combinations thereof. Among such formulations, the carrier vehicle for the hydrophobic substance will contain at least one cosolvent at a concentration of from about 5% to about 95% on a weight/weight basis. Preferred formulations will contain at least one cosolvent at a concentration of at least about 10%, at least about 20%, at least about 30%, at least about 40% up to about 50% or greater in an aqueous medium, on a weight/weight basis. Mixtures of cosolvents can also be used.

Surface active agents, i.e. surfactants, can also be present in the formulation as solubilizing agents. Such surfactants can be anionic, cationic, zwitterionic or nonionic (See for example, Yalkowisky, supra, pp. 236-320). Non-limiting examples of suitable surfactants include phospholipids such as lecithin, benzalkonium chloride, benzethonium chloride, cetylpyridinium chloride, dioctyl sodium sulfosuccinate, nonoxynol 9, nonoxynol 10, octoxynol 9, poloxamers, polyoxyethylene (8), caprylic/capric mono- and diglycerides (e.g., Labrasol™ of Gattefossé), polyoxyethylene (35) castor oil, polyoxyethylene (20) cetostearyl ether, polyoxyethylene (40) hydrogenated castor oil, polyoxyethylene (10) oleyl ether, polyoxyethylene (40) stearate, polysorbate 20, polysorbate 40, polysorbate 60, polysorbate 80 (e.g., Tween™ 80 of ICI), propylene glycol laurate (e.g., Lauroglycol™ of Gattefossé), sodium lauryl sulfate, sorbitan monolaurate, sorbitan monooleate, sorbitan monopalmitate, sorbitan monostearate, tyloxapol, and mixtures thereof.

Formulations containing surfactants comprise, preferably, from about 1% or less up to about 15% surfactant on a weight/weight basis. Preferred surfactant concentrations are at least about 2%, at least about 3%, at least about 4%, up to about 5% on a weight/weight basis.

The hydrophobic substance can also be in the form of nanoparticles or nanocrystals dispersed or suspended in an aqueous medium. In such a formulation the hydrophobic substance is nanoparticulate, i.e., having D₉₀ less than about 1 μm (D₉₀ being a diameter such that 90% by weight of the particles are smaller than this diameter in their longest dimension). In such nanoparticulate formulations, weight average particle size is typically about 100 nm to about 800 run, for example about 150 nm to about 600 nm, or about 200 nm to about 400 nm. The nanoparticles can also have a D₂₅ particle size of about 450 nm to about 1000 nm, and more preferably about 500 nm to about 900 nm D₂₅ being a diameter such that 25% by weight of the particles are smaller than this diameter in their longest dimension). Pharmaceutical compositions comprising any of such nanoparticulate formulations of the hydrophobic substance can be useful in methods of the present invention.

Numerous processes for preparation of nanoparticulate compositions of therapeutic agents are known. Some of these processes use mechanical means, such as milling, to reduce particle size to a nano range, and others precipitate nano-sized particles from solution. Illustrative processes are disclosed in the patent publications cited below.

U.S. Pat. No. 4,826,689 to Violanto & Fischer.

U.S. Pat. No. 5,145,684 to Liversidge et al.

U.S. Pat. No. 5,298,262 to Na & Rajagopalan.

U.S. Pat. No. 5,302,401 to Liversidge et al.

U.S. Pat. No. 5,336,507 to Na & Rajagopalan.

U.S. Pat. No. 5,340,564 to Illig & Sarpotdar.

U.S. Pat. No. 5,346,702 to Na & Rajagopalan.

U.S. Pat. No. 5,352,459 to Hollister et al.

U.S. Pat. No. 5,354,560 to Lovrecich.

U.S. Pat. No. 5,384,124 to Courteille et al.

U.S. Pat. No. 5,429,824 to June.

U.S. Pat. No. 5,503,723 to Ruddy et al.

U.S. Pat. No. 5,510,118 to Bosch et al.

U.S. Pat. No. 5,518,187 to Bruno et al.

U.S. Pat. No. 5,518,738 to Eickhoff et al.

U.S. Pat. No. 5,534,270 to De Castro.

U.S. Pat. No. 5,536,508 to Canal et al.

U.S. Pat. No. 5,552,160 to Liversidge et al.

U.S. Pat. No. 5,560,931 to Eickhoff et al.

U.S. Pat. No. 5,560,932 to Bagchi et al.

U.S. Pat. No. 5,565,188 to Wong et al.

U.S. Pat. No. 5,569,448 to Wong et al.

U.S. Pat. No. 5,571,536 to Eickhoff et al.

U.S. Pat. No. 5,573,783 to Desieno & Stetsko.

U.S. Pat. No. 5,580,579 to Ruddy et al.

U.S. Pat. No. 5,585,108 to Ruddy et al.

U.S. Pat. No. 5,587,143 to Wong.

U.S. Pat. No. 5,591,456 to Franson et al.

U.S. Pat. No. 5,622,938 to Wong.

U.S. Pat. No. 5,662,883 to Bagchi et al.

U.S. Pat. No. 5,665,331 to Bagchi et al.

U.S. Pat. No. 5,718,919 to Ruddy et al.

U.S. Pat. No. 5,747,001 to Wiedmann et al.

International Patent Publication No. WO 93/25190.

International Patent Publication No. WO 96/24336.

International Patent Publication No. WO 97/14407.

International Patent Publication No. WO 98/35666.

International Patent Publication No. WO 99/65469.

International Patent Publication No. WO 00/18374.

International Patent Publication No. WO 00/27369.

International Patent Publication No. WO 00/30615.

One of ordinary skill in the art will readily adapt the processes therein described to preparation of the hydrophobic substance in nanoparticulate form.

Illustrative examples are described below.

### EXAMPLE 1

This example illustrates the improved systemic absorption of (R)-5,6-dihydro-5-(methylamino)-4H-imidazo[4,5-ij]-quinoline-2(1H)-thione upon administration to the dermis.

The compound (R)-5,6-dihydro-5-(methylamino)-4H-imidazo[4,5-ij]-quinoline-2(1H)-thione has an estimated logP value of 1.62 using logKow software (Syracuse Research Corporation, North Syracuse, N.Y. 13212; see also Meylan and Howard, supra). In accordance with the invention herein, this compound is shown to give higher plasma levels and improved pharmacokinetic parameters upon administration to the dermis than that produced upon subcutaneous administration.

Six Yucatan mini-pigs weighing 20-25 kg were used. Solutions of (R)-5,6-dihydro-5-(methylamino)-4H-imidazo[4,5-ij]-quinoline-2(1H)-thione were prepared at 10 mg/mL concentrations at pH 5.5 in citrate/phosphate buffer with sufficient dextrose to achieve isotonicity received (R)-5,6-dihydro-5-(methylamino)-4H-imidazo[4,5-ij]-quinoline-2(1H)-thione by the following routes: (A) intravenous bolus, (B) subcutaneous injection, and (C) injection to the dermis by a microneedle array. A total of 6 animals were utilized in a full crossover design where each animal received a 1.0 mg dose by all administration routes in an injection volume of 0.1 mL.

The intravenous dose was administered an ear vein catheter. Subcutaneous delivery was through a standard 0.5 inch, 30 gauge needle. Delivery to the dermis was through a three point microneedle array having three 34 gauge needles with 7 mm spacing and 1 mm depth to right flank of the animal between the rib cage and the rear leg. Subcutaneous and intravenous administration was by manual injection seconds. Administration to the dermis was at a rate of 90 μL/min by a syringe pump.

The study design was a full crossover with each animal receiving intravenous, subcutaneous and dermal administration. Each animal received three treatments over a two-week period with a minimal washout period of 2 days between subsequent doses. Dosing was according to the schedule shown below in Table 2.

TABLE 2
Dosing Schedule*

| | Week 1 | | Week 2 | | Week 3 | | Week 4 | | Week 5 |
|---|---|---|---|---|---|---|---|---|---|
| Animal | Dose 1 | Dose 2 | Dose 3 | Dose 4 | Dose 5 | Dose 6 | Dose 7 | Dose 8 | Dose 9 |
| 1 | IV | ID | SC | | | | | | |
| 2 | ID | SC | IV | | | | | | |
| 3 | | | | SC | IV | ID | | | |
| 4 | | | | IV | ID | SC | | | |
| 5 | | | | | | | ID | SC | IV |
| 6 | | | | | | | SC | IC | ID |

*IV represents intravenous administration, SC represents subcutaneous administration and ID represents administration to the dermis.

Blood samples were obtained from the vena cava access port immediately before dosing and at 5, 10, 15, 20, 30, 45, 60 minutes and 2, 3, 4, 6, 8, 10, 14 and 24 hours after administration. Timing was started at the cessation of injection for a given method. The venous samples were collected into EDTA containing Vacutainer tubes, centrifuged at approximately 1000 g for 10 minutes at 4° C. After centrifugation, the plasma layer was transferred to plastic storage vials and stored frozen at −70° C. until assayed.

Assay of the pig plasma samples was performed by HPLC analysis. Results are shown below in Tables 3

TABLE 3
Plasma Concentrations (ng/mL) of (R)-5,6-dihydro-5-
(methylamino)-4H-imidazo[4,5-ij]-quinoline-2(1H)-thione
Following Administration By Various Routes In
Yucatan Mini-pigs.

| | | |
|---|---|---|
| IV* | ID | SC |

| TIME | Mean | SD | Mean | SD | Mean | SD |
|---|---|---|---|---|---|---|
| 5 min | 29.8 | 7.0 | 24.8 | 12.4 | 10.0 | 2.2 |
| 10 min | 20.5 | 4.5 | 22.5 | 9.0 | 15.3 | 3.0 |
| 15 min | 18.5 | 4.2 | 16.4 | 6.2 | 16.0 | 1.7 |
| 20 min | 15.3 | 2.2 | 18.0 | 6.6 | 14.5 | 1.8 |
| 30 min | 13.1 | 2.4 | 14.0 | 2.4 | 14.0 | 0.9 |
| 45 min | 10.7 | 2.3 | 9.9 | 2.7 | 9.9 | 3.1 |
| 1 hr | 9.1 | 1.5 | 9.0 | 1.6 | 8.9 | 1.4 |
| 2 hr | 6.7 | 1.4 | 5.7 | 1.9 | 5.6 | 1.4 |
| 3 hr | 2.8 | 2.6 | 4.2 | 1.5 | 4.1 | 2.0 |
| 4 hr | 1.6 | 1.5 | 3.6 | 1.4 | 2.2 | 1.9 |
| 6 hr | 2.6 | 4.1 | 0.4 | 1.0 | 1.6 | 3.3 |
| 8 hr | 0 | 0 | 0 | 0 | 0.6 | 1.6 |
| 10 hr | 0 | 0 | 0 | 0 | 0 | 0 |
| 14 hr | 0 | 0 | 0 | 0 | 0 | 0 |
| 24 hr | 0 | 0 | 0 | 0 | 0 | 0 |

*Drug was given by intravenous administration (IV), subcutaneous administration (SC) and by administration to the dermis (ID). The intravenous dose was administered to the 1.0 mg dosing subgroup into an ear vein.

As shown in Tables 3, administration into the dermis tended to produce higher plasma levels of drug than that produce upon subcutaneous administration. Analysis of the data by Heterogeneity of Regression analysis revealed that values obtained following administration to the dermis and intravenous administration were not different from each other, but different from values obtained following subcutaneous administration.

Pharmacokinetic parameters were estimated using Watson Drug Metabolism Laboratory Information Management System, version 6.2.0.02 (Innaphase Corp., Philadelphia, Pa.). Parameters determined were the maximal plasma concentration, Cₘₐₓ, time to maximal plasma concentration Tₘₐₓ, area under the curve estimated to t=infinity, Tₘₐₓ and the half-life for decrease in plasma concentration, T_{1/2}.

Pharmacokinetic parameters are shown in Table 4 below.

TABLE 4
Mean Pharmacokinetic Parameters (±standard deviation)
of (R)-5,6-dihydro-5-(methylamino)-4H-imidazo[4,5-ij]-
quinoline-2(1H)-thione Following Administration By Various
Routes^{a} In Yucatan Mini-pigs.

| | 1.0 mg Dose | | |
|---|---|---|---|
| | IV | ID | SC |
| PARAMETER | (n = 6) | (n = 6) | (n = 6) |
| Dose (μg/kg) | 40.9 | 41.3 | 40.3 |
| | (3.2) | (3.9) | (3.4) |
| Cₘₐₓ (ng/mL) | 29.8 | 26.7 | 16.9 |
| | (7.0) | (10.2) | (2.6) |
| Tₘₐₓ (hr) | 0.083 | 0.139 | 0.222 |
| | (0.000) | (0.101) | (0.068) |
| AUC_{(0-∞)} (ng * hr/mL) | 32.4 | 33.6 | 32.1 |
| | (8.2) | (9.0) | (9.2) |
| T_{1/2} (hr) | 1.45 | 1.61 | 1.5 |
| | (0.38) | (0.70) | (0.53) |

^{a}Drug was given by intravenous administration (IV), subcutaneous administration (SC) and by administration to the dermis (ID).

As shown in the table, the Cₘₐₓ values were consistently greater and the Tₘₐₓ values were consistently lesser following administration to the dermis than values for the same parameters obtained following subcutaneous administration of the hydrophobic substance, (R)-5,6-dihydro-5-(methylamino)-4H-imidazo[4,5-ij]-quinoline-2(1H)-thione.

As various changes could be made in the above methods and compositions without departing from the scope of the invention, it is intended that all matter contained in the above description be interpreted as illustrative and not in a limiting sense.

All references cited in this specification are hereby incorporated by reference. The discussion of the references herein is intended merely to summarize the assertions made by their authors and no admission is made that any reference constitutes prior art relevant to patentability. Applicant reserves the right to challenge the accuracy and pertinency of the cited references.

What is claimed is:

1. A method for systemic administration of a hydrophobic dopamine agonist having a logP of about 1.5 or greater to a mammal, the method comprising delivering the dopamine agonist by bolus injection to the dermis of the mammal, wherein increased systemic absorption is produced as compared to absorption produced upon delivering the dopamine agonist subcutaneously by bolus injection.

2. The method of claim 1 wherein the dopamine agonist is as set forth in formula (I): 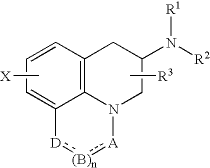
(I) or pharmaceutically acceptable salts thereof, wherein
R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;
X is H, F, Cl, Br, I, OH, C₁₋₆ alkyl or alkoxy, CN, carboxamide, carboxyl or (C₁₋₆ alkyl)carbonyl;
A is CH, CH₂, CHF, CHCl, CHBr, CHI, CHCH₃, C═O, C═S, CSCH₃, C—NH, CNH₂, CNHCH₃, CNHCOOCH₃, CNHCN, SO₂ or N;
B is CH, CH₂, CHF, CHCl, CHBr, CHI, C═O, N, NH or NCH₃, and n is 0 or 1; and
D is CH, CH₂, CHF, CHCl, CHBr, CHI, C═O, O, N, NH or NCH₃; and n is 0 or 1, and where [symbol] is a single or double bond, with the provisos that:
(1) when n is 0, and A is CH₂, CH-(halogen) where halogen is defined above, CHCH₃, C═O, C═S, C—NH, SO₂;
then D is CH₂, CH-(halogen) where halogen is as defined above, C═O, O, NH, N—CH₃;
(2) when n is 0, and
A is CH, C—SCH₃, C—NH₂, C—NHCH₃, C—NHCOOCH₃, C—NHCN, N; then
D is CH, N;
(3) when n is 1, and
A is CH₂, CH-(halogen) where halogen is as defined above, CHCH₃, C═O, C═S, C═NH, SO₂; and
B is CH₂, CH-(halogen) where halogen is as defined above, C═O, NH, N—CH₃; then
D is CH₂, C═O, O, NH, N—CH₃;
(4) when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) when n is l, and
A is CH₂, CHCH₃, C═O, C═, C═NH, SO₂, and
B is CH, N; then
D is CH, N; and pharmaceutically acceptable salts thereof to the human.

3. The method of claim 2 wherein the dopamine agonist is (R)5,6-dihydro-5-(methylamino)-4H-imidazo[4,5-ij]-quinoline-2(1H)-thione or a pharmaceutically acceptable salt thereof.

4. The method of claim 2 wherein the dopamine agonist is (R)-5,6-dihydro-5-(methylamino)-4H-imidazo[4,5-ij]quinolin-2(1H)-one or a pharmaceutically acceptable salt thereof.

5. The method of claim 1 wherein the bolus subcutaneous injection is delivered in not more than 10 minutes.

6. The method of claim 5 wherein the bolus subcutaneous injection is delivered in not more than 2 minutes.

7. The method of claim 1 wherein the hydrophobic dopamine agonist is delivered to the dermis in not more than 10 minutes.

8. The method of claim 7 wherein the hydrophobic dopamine agonist is delivered to the dermis in not more than 2 minutes.

9. The method of claim 1 wherein the hydrophobic dopamine agonist is delivered to the dermis by repeated bolus injection.

10. The method of claim 1 wherein at least one pharmacokinetic parameter is improved upon delivery of the dopamine agonist to the dermis as compared to the same pharmacokinetic parameter upon delivering the dopamine agonist subcutaneously by bolus injection.

11. The method of claim 10, wherein the improved pharmacokinetic parameter comprises increased bioavailability of the dopamine agonist.

12. The method of claim 10, wherein the improved pharmacokinetic parameter comprises a decrease in Tₘₐₓ.

13. The method of claim 10, wherein the improved pharmacokinetic parameter comprises an increase in Cₘₐₓ.

14. The method of claim 10, wherein the improved pharmacokinetic parameter comprises a decrease in T_{lag}.

15. The method of claim 1 wherein the delivering is through a cutaneous micropore created by any solid projection, electromotive force, thermal energy or gas ballistics.

16. The method of claim 15 wherein the delivering is through at least one hollow needle.

17. The method of claim 16 wherein the at least one hollow needle comprises an array of microneedles.

18. The method of claim 17 wherein the dopamine agonist is delivered by infusion pump, piezoelectric pump, electromotive pump, electromagnetic pump, Belleville spring, iontophoresis, or sonophoresis.

19. The method of claim 1 wherein the hydrophobic dopamine agonist has a logP of about 2.0 or greater.

20. The method of claim 1 wherein the dopamine agonist is in the form of nanoparticles or nanocrystals.

21. A method for administration of a hydrophobic dopamine agonist having a logP of about 1.5 or greater to a mammal, the method comprising selectively delivering the dopamine agonist by bolus injection to the dermis of the mammal to obtain systemic absorption of the dopamine agonist from the dermis.

22. The method of claim 21 wherein increased systemic absorption of the dopamine agonist is produced upon delivering the dopamine agonist to the dermis by bolus injection as compared to absorption produced upon delivering the dopamine agonist subcutaneously by bolus injection.

23. The method of claim 22 wherein the dopamine agonist is as set forth in formula (I): 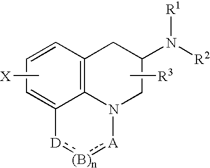
(I) or pharmaceutically acceptable salts thereof, wherein
R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;
X is H, F, Cl, Br, I, OH, C₁₋₆ alkyl or alkoxy, CN, carboxamide, carboxyl or (C₁₋₆ alkyl)carbonyl;
A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;
B is CH, CH₂, CHF, CHCl, CHBr, CM, C═O, N, NH or NCH₃, and n is 0 or 1; and
D is CH, CH₂, CHF, CHCl, CHBr, CHI, C═O, N, NH or NCH₃; and n is 0 or 1, and where is a single or double bond,
with the provisos that:
(1) when n is 0, and A is CH₂, CH-(Halogen) where halogen is defined above, CHCH₃, C═O, C═, C—NH, SO₂;
then D is CH₂, CH-(halogen) where halogen is as defined above, C═O, O, NH, N—CH₃;
(2) when n is 0, and
A is CH, C—SCH₃, C—NH₂, C—NHCH₃, C—NHCOOCH₃, C—NHCN, N; then
D is CH, N;
(3) when n is 1, and
A is CH₂, CH-(halogen) where halogen is as defined above, CHCH₃, C═O, C═S, C—NH, SO₂; and
B is CH₂, CH-(halogen) where halogen is as defined above, C═O, NH, N—CH₃; then
D is CH₂, C═O, O, NH, N—CH₃;
(4) when n is 1, and
A is CH, C—CH₃, C—NH₂, C—NHCH₃, C—NHCOOCH₃, C—NHCN, N; and
B is CH, N; then
D is CH₂, C═O, O, NH, N—CH₃;
(5) when n is 1, and
A is CH₂, CHCH₃, C═O, C═S, C═NH, SO₂, and
B is CH, N; then
D is CH, N; and pharmaceutically acceptable salts thereof to the human.

24. The method of claim 23 wherein the dopamine agonist is (R)-5,6-dihydro-5-(methylamino)-4H-imidazo[4,5-ij]-quinoline-2(1H)-thione or a pharmaceutically acceptable salt thereof.

25. The method of claim 23 wherein the dopamine agonist is (R)-5,6-dihydro-5-(methylamino)-4H-imidazo[4,5-ij]-quinolin-2(1H)-one or a pharmaceutically acceptable salt thereof.

26. The method of claim 22 wherein the bolus subcutaneous injection is delivered in not more than 10 minutes.

27. The method of claim 26 wherein the bolus subcutaneous injection is delivered in not more than 2 minutes.

28. The method of claim 22 wherein the hydrophobic dopamine agonist is delivered to the dermis in not more than 10 minutes.

29. The method of claim 28 wherein the hydrophobic dopamine agonist is delivered to the dermis in not more than 2 minutes.

30. The method of claim 22 wherein the hydrophobic dopamine agonist is delivered to the dermis by repeated bolus injection.

31. The method of claim 22 wherein at least one pharmacokinetic parameter is improved upon delivery of the dopamine agonist to the dermis as compared to the same pharmacolietic parameter upon delivering the dopamine agonist subcutaneously by bolus injection.

32. The method of claim 31, wherein the improved pharmacokinetic parameter comprises increased bioavailability of the dopamine agonist.

33. The method of claim 31, wherein the improved pharmacokinetic parameter comprises a decrease in Tₘₐₓ.

34. The method of claim 31, wherein the improved pharmacokinetic parameter comprises an increase in Cₘₐₓ.

35. The method of claim 31, wherein the improved pharmacokinetic parameter comprises a decrease in T_{lag}.

36. The method of claim 22 wherein the delivering is through a cutaneous micropore created by any solid projection, electromotive force, thermal energy or gas ballistics.

37. The method of claim 22 wherein the delivering is through at least one hollow needle.

38. The method of claim 37 wherein the at least one hollow needle comprises an array of microneedles.

39. The method of claim 37 wherein the dopamine agonist is delivered by infusion pump, piezoelectric pump, electromotive pump, electromagnetic pump, Belleville spring, iontophoresis, or sonophoresis.

40. The method of claim 22 wherein the hydrophobic dopamine agonist has a logP of about 2.0 or greater.

41. The method of claim 22 wherein the dopamine agonist is in the form of nanoparticles or nanocrystals.

42. A method for administration of a hydrophobic dopamine agonist have a logP of about 1.5 or greater to a mammal, the method comprising selectively delivering the dopamine agonist by bolus injection to the dermis of the mammal wherein systemic absorption of the dopamine agonist from the dermis is obtained.

43. The method of claim 42 wherein increased systemic absorption of the dopamine agonist is produced upon delivering the dopamine agonist by bolus injection to the dermis as compared to absorption produced upon delivering the dopamine agonist subcutaneously by bolus injection.

44. The method of claim 43 wherein the dopamine agonist is as set forth in formula (I): 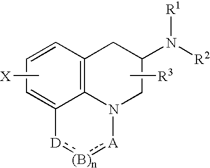
(1) or pharmaceutically acceptable salts thereof, wherein
R¹, R² and R³ are the same or different and are H, C₁ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;
X is H, F, Cl, Br, I OH, C₁₋₆ alkyl or alkoxy, CN, carboxamide, carboxyl or (C₁₋₆ alkyl)carbonyl;
A is CH, CH₂, CHF, CHCl, CHBr, CHI, CHCH₃, C═O, C—S, CSCH₃, C═NH, CNH₂, CNHCH₃, CNHCOOCH₃, CNHCN, SO₂ or N;
B is CH, CH₂, CHF, CHCl, CHBr, CHI, C═O, N, NH or NCH₃, and n is 0 or 1; and
D is CH, CH₂, CHF, CHCl, CHBr, CHI, C═O, O, N, NH or NCH₃; and n is 0 or 1, and where [symbol] is a single or double bond,
with the provisos that:
(1) when n is 0, and A is CH₂, CH-(halogen) where halogen is defined above, CHCH₃, C═O, C═S, C═NH, SO₂;
then D is CH₂, CH-(halogen) where halogen is as defined above, C═O, O, NH, N—CH₃;
(2) when n is 0, and
A is CH, C—SCH₃, C—NH₂, C—NHCH₃, C—NHCOOCH₃, C—NHCN, N; then
D is CH, N;
(3) when n is 1, and
A is CH₂, CH-(halogen) where halogen is as defied above, CHCH₃, CO, C═S, C═NH, SO₂; and
B is CH₂, CH-(halogen) where halogen is as defined above, C═O, NH, N—CH₃; then
D is CH₂, C═O, O, NH, N—CH₃;
(4) when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) when n is 1, and
A is CH₂, CHCH₃, C═O, C—S, C—NH, SO₂, and
B is CH, N; then
D is CH, N; and pharmaceutically acceptable salts thereof to the human.

45. The method of claim 44 wherein the dopamine agonist is (R)-5,6-dihydro-5-(methylamino)-4H-imidazo[4,5-ij]-quinoline-2(H)-thione or a pharmaceutically acceptable salt thereof.

46. The method of claim 44 wherein the dopamine agonist is (R)-5,6-dihydro-S-(methylamino)-4H-imidazo[4,5-ij]-quinolin-2(1H)-one or a pharmaceutically acceptable salt thereof.

47. The method of claim 43 wherein the bolus subcutaneous injection is delivered in not more than 10 minutes.

48. The method of claim 47 wherein the bolus subcutaneous injection is delivered in not more than 2 minutes.

49. The method of claim 43 wherein the hydrophobic dopamine agonist is delivered to the dermis in not more than 10 minutes.

50. The method of claim 49 wherein the hydrophobic dopamine agonist is delivered to the dermis in not more than 2 minutes.

51. The method of claim 43 wherein the hydrophobic dopamine agonist is delivered to the dermis by repeated bolus injection.

52. The method of claim 43 wherein at least one pharmacokinetic parameter is improved upon delivery of the dopamine agonist to the dermis as compared to the same pharmacokinetic parameter upon delivering the dopamine agonist subcutaneously by bolus injection.

53. The method of claim 52, wherein the improved pharmacokinetic parameter comprises increased bioavailability of the dopamine agonist.

54. The method of claim 52, wherein the improved pharmacokinetic parameter comprises a decrease in Tₘₐₓ.

55. The method of claim 52, wherein the improved pharmacokinetic parameter comprises an increase in Cₘₐₓ.

56. The method of claim 52, wherein the improved pharmacokinetic parameter comprises a decrease in T_{lag}.

57. The method of claim 43 wherein the delivering is through a cutaneous micropore created by any solid projection, electromotive force, thermal energy or gas ballistics.

58. The method of claim 43 wherein the delivering is through at least one hollow needle.

59. The method of claim 58 wherein the at least one hollow needle comprises an array of microneedles.

60. The method of claim 58 wherein the dopamine agonist is delivered by infusion pump, piezoelectric pump, electromotive pump, electromagnetic pump, Bellevi611le spring, iontophoresis, or sonophoresis.

61. The method of claim 43 wherein the hydrophobic dopamine agonist has a logP of about 2.0 or greater.

62. The method of claim 43 wherein the dopamine agonist is in the form of nanoparticles or nanocrystals.

63. A method for administration of a hydrophobic dopamine agonist having a logP of about 1.5 or greater to a mammal, the method comprising selectively delivering the dopamine agonist by bolus injection to the dermis of the mammal to achieve a substantially higher bioavailability and/or a substantially higher Cₘₐₓ and/or a substantially shorter Tₘₐₓ and/or a substantially shorter T_{lag}, and/or a substantially greater Kₐ as compared to that produced upon bolus subcutaneous administration of the dopamine agonist at an identical dose.

64. The method of claim 63 wherein the dopamine agonist is as set forth in formula (I): 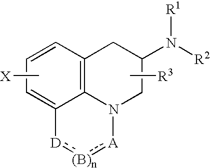
(I) or pharmaceutically acceptable salts thereof, wherein
R¹, R² and R³ are the same or different and are H, C₁₋₆ alkyl (optionally phenyl substituted), C₃₋₅ alkenyl or alkyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;
X is H, F, Cl, Br, I, OH, C₁₋₆ alkyl or alkoxy, CN, carboxamide, carboxyl or (C₁₋₆ alkyl)carbonyl;
A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;
B is CH, CH₂, CHF, CHCl, CHBr, CHI, C═O, N, NH or NCH₃, and n is 0 or 1; and
D is CH, CH₂, CHF, CHCl, CHBr, CHI, C═O, O, N, NH or NCH₃; and n is 0 or 1, and where [symbol] is a single or double bond, with the provisos that:
(1) when n is 0, and A is CH₂, CH-(halogen) where halogen is defined above, CHCH₃, C═O, O═S, C═NH, SO₂;
then D is CH₂, CH-(halogen) where halogen is as defined above, C═O, O, NH, N—CH_{3x;}
(2) when n is 0, and
A is CH, C—SCH₃, C—NH₂, C—NHCH₃, C—NHCOOCH₃, C—NHCN, N; then
D is CH, N;
(3) when n is 1, and
A is CH₂, CH-(halogen) where halogen is as defined above, CHCH₃, C═O, C═S, C═NH, SO₂; and
B is CH₂, CH-(halogen) where halogen is as defined above, C═O, NH, N—CH₃; then
D is CH₂, C═O, O, NH, N—CH₃;
(4) when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) when n is 1, and
A is CH₂, CHCH₃, C═O, C═S, C═NH, SO₂, and
B is CH, N; then
D is CH, N; and pharmaceutically acceptable salts thereof to the human.

65. The method of claim 64 wherein the dopamine agonist is (R)-5,6-dihydro-5-(methylamino)-4H-imidazo[4,5-ij]-quinoline-2(1H)-thione or a pharmaceutically acceptable salt thereof.

66. The method of claim 64 wherein the dopamine agonist is (R)-5,6-dihydro-5-(methylamino)-4H-imidazo[4,5-ij]-quinolin-2(1H)-one or a pharmaceutically acceptable salt thereof.

67. The method of claim 63 wherein the bolus subcutaneous injection is delivered in not more than 10 minutes.

68. The method of claim 67 wherein the bolus subcutaneous injection is delivered in not more than 2 minutes.

69. The method of claim 63 wherein the hydrophobic dopamine agonist is delivered to the dermis in not more than 10 minutes.

70. The method of claim 69 wherein the hydrophobic dopamine agonist is delivered to the dermis in not more than 2 minutes.

71. The method of claim 63 wherein the hydrophobic dopamine agonist is delivered to the dermis by repeated bolus injection.

72. The method of claim 63 wherein at least one pharmacokinetic parameter is improved upon delivery of the dopamine agonist to the dermis as compared to the same pharmacokinetic parameter upon delivering the dopamine agonist subcutaneously by bolus injection.

73. The method of claim 72, wherein the improved pharmacokinetic parameter comprises increased bioavailability of the dopamine agonist.

74. The method of claim 72, wherein the improved pharmacokinetic parameter comprises a decrease in Tₘₐₓ.

75. The method of claim 72, wherein the improved pharmacokinetic parameter comprises an increase in Cₘₐₓ.

76. The method of claim 72, wherein the improved pharmacokinetic parameter comprises a decrease in T_{lag}.

77. The method of claim 63 wherein the delivering is through a cutaneous micropore created by any solid projection, electromotive force, thermal energy or gas ballistics.

78. The method of claim 63 wherein the delivering is through at least one hollow needle.

79. The method of claim 78 wherein the at least one hollow needle comprises an array of microneedles.

80. The method of claim 78 wherein the dopamine agonist is delivered by infusion pump, piezoelectric pump, electromotive pump, electromagnetic pump, Belleville spring, iontophoresis, or sonophoresis.

81. The method of claim 63 wherein the hydrophobic dopamine agonist has a logP of about 2.0 or greater.

82. The method of claim 63 wherein the dopamine agonist is in the form of nanoparticles or nanocrystals.

83. A method for administration of a hydrophobic dopamine agonist having a logP of about 1.5 or greater to a mammal, the method comprising selectively delivering the dopamine agonist by bolus injection to the dermis of the mammal wherein a substantially higher bioavailability and/or a substantially higher Cₘₐₓ and/or a substantially shorter Tₘₐₓ and/or a substantially shorter T_{lag}, and/or a substantially greater Kₐ is produced as compared to that produced upon bolus subcutaneous administration of the dopamine agonist at an identical dose.

84. The method of claim 83 wherein the dopamine agonist is as set forth in formula (I): 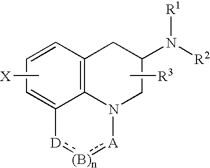
(I) or pharmaceutically acceptable salts thereof, wherein
R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;
X is H, F, Cl, Br, I, OH, C₁₋₆ alkyl or alkoxy, CN, carboxamide, carboxyl or (C₁₋₆ allyl)carbonyl;
A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;
B is CH, CH₂, CHF, CHCl, CHBr, CHI, C═O, N, NH or NCH₃, and n is 0 or 1; and
D is CH, CH₂, CHF, CHCl, CHBr, CHI, C═O, O, N, NH or NCH₃; and n is 0 or 1, and where [symbol] is a single or double bond,
with the provisos that:
(1) when n is 0, and A is CH₂, CH-(halogen) where halogen is defined above, CHCH₃, C═O, C═S, C═NH, SO₂;
then D is CH₂, CH-(halogen) where halogen is as defined above, C═O, O, NH, N—CH₃;
(2) when n is 0, and
A is CH, C—SCH₃, C—NH₂, C—NHCH₃, C—NHCOOCH₃, C—NHCN, N; then
D is CH, N;
(3) when n is 1, and
A is CH₂, CH-(halogen) where halogen is as defined above, CHCH₃, C═O, C═S, C═NH, SO₂; and
B is CH₂, CH-(halogen) where halogen is as defined above, C═O, NH, N—CH₃; then
D is CH₂, C═O, O, NH, N—CH₃;
(4) when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) when n is 1, and
A is CH₂, CHCH₃, C═O, S, C—NH, SO₂, and
B is CH, N; then
D is CH, N; and pharmaceutically acceptable salts thereof to the human.

85. The method of claim 84 wherein the dopamine agonist is (R)-5,6-dihydro-5-(methylamino)-4H-imidazo[4,5-ij]-quinoline-2(1H)-thione or a pharmaceutically acceptable salt thereof.

86. The method of claim 84 wherein the dopamine agonist is (R)-5,6-dihydro-5-(methylamino)-4H-imidazo[4,5-ij]-quinolin-2(1H)-one or a pharmaceutically acceptable salt thereof.

87. The method of claim 83 wherein the bolus subcutaneous injection is delivered in not more than 10 minutes.

88. The method of claim 87 wherein the bolus subcutaneous injection is delivered in not more than 2 minutes.

89. The method of claim 83 wherein the hydrophobic dopamine agonist is delivered to the dermis in not more than 10 minutes.

90. The method of claim 89 wherein the hydrophobic dopamine agonist is delivered to the dermis in not more than 2 minutes.

91. The method of claim 83 wherein the hydrophobic dopamine agonist is delivered to the dermis by repeated bolus injection.

92. The method of claim 83 wherein at least one pharmacokinetic parameter is improved upon delivery of the dopamine agonist to the dermis as compared to the same pharmacokinetic parameter upon delivering the dopamine agonist subcutaneously by bolus injection.

93. The method of claim 92, wherein the improved pharmacokinetic parameter comprises increased bioavailability of the dopamine agonist.

94. The method of claim 92, wherein the improved pharmacokinetic parameter comprises a decrease in Tₘₐₓ.

95. The method of claim 92, wherein the improved pharmacokinetic parameter comprises an increase in Cₘₐₓ.

96. The method of claim 92, wherein the improved pharmacokinetic parameter comprises a decrease in T_{lag}.

97. The method of claim 83 wherein the delivering is through a cutaneous micropore created by any solid projection, electromotive force, thermal energy or gas ballistics.

98. The method of claim 83 wherein the delivering is through at least one hollow needle.

99. The method of claim 98 wherein the at least one hollow needle comprises an array of microneedles.

100. The method of claim 98 wherein the dopamine agonist is delivered by infusion pump, piezoelectric pump, electromotive pump, electromagnetic pump, Belleville spring, iontophoresis, or sonophoresis.

101. The method of claim 83 wherein the hydrophobic dopamine agonist has a logP of about 2.0 or greater.

102. The method of claim 83 wherein the dopamine agonist is in the form of nanoparticles or nanocrystals.
